SEQUENCE LISTING (11 sequences)

SEQ ID NO: 1 - protein, 5 aa, Artificial Sequence
  Artificially Synthesized Sequence
NYWMH

SEQ ID NO: 2 - protein, 17 aa, Artificial Sequence
  Artificially Synthesized Sequence
ATYRGHSDTYYNQKFKG

SEQ ID NO: 3 - protein, 12 aa, Artificial Sequence
  Artificially Synthesized Sequence
GAIYDGYDVLDN

SEQ ID NO: 4 - protein, 11 aa, Artificial Sequence
  Artificially Synthesized Sequence
SASQDISNYLN

SEQ ID NO: 5 - protein, 7 aa, Artificial Sequence
  Artificially Synthesized Sequence
YTSNLHS

SEQ ID NO: 6 - protein, 9 aa, Artificial Sequence
  Artificially Synthesized Sequence
QQYRKLPWT

SEQ ID NO: 7 - protein, 121 aa, Artificial Sequence
  Artificially Synthesized Sequence
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 8 - protein, 108 aa, Artificial Sequence
  Artificially Synthesized Sequence
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 9 - protein, 451 aa, Artificial Sequence
  Artificially Synthesized Sequence
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  Artificially Synthesized Sequence
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 184 aa, Homo Sapien
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

(19) United States
(12) Patent Application Publication
(10) Pub. No.: US 2022/0003772 A1
(43) Pub. Date: Jan. 6, 2022

(54) METHODS OF TREATING CANCER

(71) Applicant: GLAXOSMITHKLINE INTELLECTUAL PROPERTY DEVELOPMENT LIMITED, Brentford (GB)

(72) Inventors: Elisha J. DETTMAN, Collegeville, PA (US); Joanna OPALINSKA, Collegeville, PA (US)

(21) Appl. No.: 17/289,389

(22) PCT Filed: Oct. 29, 2019
(86) PCT No.: PCT/IB2019/059273

§ 371 Date: Apr. 28, 2021

Related U.S. Application Data
(60) Provisional application No. 62/771,325, filed on Nov. 26, 2018.
(60) Provisional application No. 62/753,191, filed on Oct. 31, 2018.

(51) Int. Cl. G01N 33/574; A61P 35/00; C07K 16/28; A61K 39/395; A61K 45/06

(52) U.S. Cl. CPC G01N 33/57488; A61P 35/00; C07K 16/2878; A61K 2039/545; A61K 45/06; G01N 2800/52; A61K 39/39558

(57) ABSTRACT

This invention relates to methods of treating cancer in a patient need thereof, e.g., in a human subject, comprising determining the level of soluble BCMA (sBCMA) in a sample from the patient, and administering to the patient an effective amount of a BCMA antigen binding protein, thereby treating the cancer in the patient. Also provided are kits for the treatment of cancer comprising a means for determining the level of sBCMA in a sample from a patient.

### FIELD OF THE INVENTION

The present invention relates generally to the treatment of human disease, for example to the treatment of cancer. More specifically, the present invention relates to the use of soluble BCMA (sBCMA) levels in serum to identify patients more likely to respond to BCMA antigen binding proteins in the treatment of cancer.

### BACKGROUND OF THE INVENTION

Effective treatment of hyperproliferative disorders, including cancer, is a continuing goal in the oncology field. Generally, cancer results from the deregulation of the normal processes that control cell division, differentiation and apoptotic cell death and is characterized by the proliferation of malignant cells which have the potential for unlimited growth, local expansion and systemic metastasis. Deregulation of normal processes includes abnormalities in signal transduction pathways and response to factors that differ from those found in normal cells.

B-cell maturation antigen (BCMA) is a tumour necrosis superfamily cell-surface receptor required for plasma cell survival. The normal function of BCMA is to promote the survival of B cells at later stages of differentiation, including plasma cells. Mice lacking expression of BCMA demonstrate a reduced number of long-lived bone marrow plasma cells but have an otherwise normal phenotype. BCMA membrane expression is present on a subset of normal late-stage B cells and is universally detected on normal and malignant plasma cells, including multiple myeloma (MM) cells.

The expanding development and use of targeted therapies for cancer treatment reflects an increasing understanding of key oncogenic pathways, and how the targeted perturbation of these pathways corresponds to clinical response. Difficulties in predicting efficacy to targeted therapies is likely a consequence of the limited global knowledge of causal mechanisms for pathway deregulation (e.g. activating mutations, amplifications). Treating selected patient populations may help maximize the potential of a therapy. There is still a need to determine the prognosis of a patient afflicted with a B-cell disorder, such as multiple myeloma, in order to choose the optimal treatment plan for individual patients.

### SUMMARY OF THE INVENTION

The invention provides methods for diagnosing, determining the prognosis of, or optimizing treatment plans for, cancer patients by determining the presence or amount of soluble BCMA expression in a patient sample. It has been discovered that expression of soluble BCMA can be used as a biomarker to diagnose or determine the prognosis in cancer patients. Furthermore, it has been discovered that the presence or expression level of soluble BCMA in cancer patients can be used to select certain patient populations for treatment with a BCMA antigen binding protein and to inform dosing and treatment regimens by a clinician.

In one embodiment, the invention provides for a method of diagnosing cancer in a patient, comprising: (a) obtaining a sample from the patient; and (b) testing the sample for the presence of soluble BCMA expression, wherein if the patient expresses soluble BCMA or expresses sBCMA at a high level, the patient is determined to have cancer.

In one embodiment, the invention provides for a method for determining prognosis of cancer in a patient, comprising: (a) obtaining a sample from the patient; and (b) testing the sample for the presence of soluble BCMA expression; wherein, if the patient has expression of soluble BCMA, then the patient's prognosis is poor.

In another embodiment, the invention provides for a method for determining prognosis of cancer in a patient, comprising: (a) obtaining a sample from the patient; and (b) testing the sample for the level of soluble BCMA expression; wherein, if the patient has a high level of soluble BCMA expression, then the patient's prognosis is poor.

In one embodiment, the invention provides for methods of predicting a patient's response to treatment with a BCMA antigen binding protein comprising: (a) obtaining a sample from a patient; and (b) testing the sample for the level of soluble BCMA expression, wherein if the patient expresses a high level of soluble BCMA, the patient is predicted to not respond to treatment with a BCMA antigen binding protein.

In a further embodiment, the invention provides for a method for treating cancer in a patient in need thereof, comprising: (a) obtaining a sample from the patient; and (b) testing the sample for expression of soluble BCMA; and (c) if the subject expresses soluble BCMA, administering to the patient an effective amount of a BCMA antigen binding protein.

In yet another embodiment, the invention provides for a method for treating cancer in a patient in need thereof, comprising: (a) obtaining a sample from the patient; and (b) testing the sample for the level of soluble BCMA expression; and (c) if the patient has a high level of soluble BCMA expression, administering to the patient an effective amount of a BCMA antigen binding protein.

Further provided are methods for selecting the dose of a BCMA antigen binding protein for treating cancer in a patient in need thereof, comprising: (a) obtaining a sample from the patient; (b) testing the sample for the level of soluble BCMA expression; and (c) if the patient has a low level of soluble BCMA expression, treating the patient with a low dose of a BCMA antigen binding protein; or if the patient has a high level soluble BCMA expression, treating the patient with a high dose of a BCMA antigen binding protein.

In one embodiment, the patient is a human patient or human subject.

In one aspect of the invention, the cancer is selected from the group consisting of multiple myeloma, lymphoma, chronic lymphocytic leukemia, non-Hodgkin's lymphoma, follicular lymphoma, and diffuse large B-cell lymphoma.

In another aspect of the invention, the sample obtained from the patient is serum sample or a blood sample.

The invention provides for BCMA antigen binding proteins selected from an antibody, an antibody fragment, a bispecific antibody, an antibody-drug-conjugate, a bispecific T-cell engager (BITE), and a chimeric antigen receptor T-cell (CAR-T).

In one aspect of the invention, the BCMA antigen binding protein is a monoclonal antibody comprising a VH comprising an amino acid sequence set forth in SEQ ID NO:7; and a VL comprising an amino acid sequence set forth in SEQ ID NO:8, wherein the antibody is conjugated to MMAF.

In certain aspects, in addition to the BCMA antigen binding protein, the patient is further treated with at least one additional anti-neoplastic agent. In one embodiment, the at least one additional neoplastic agent is selected from the group consisting of an anti-PD1 antibody (e.g. nivolumab or pembrolizumab), an anti-ICOS antibody, and anti-OX40 antibody, an anti-CD38 antibody (e.g. daratumumab) a proteasome inhibitor (e.g. bortezomib, carfilzomib, or ixazomib), a thalidomide analog (e.g. lenalidomide or pomalidomide), and dexamethasone.

The invention further provides for a BCMA antigen binding protein for use in the treatment of cancer in a patient, wherein the patient is characterized by a high level of soluble BCMA expression in a sample from the patient. In another embodiment, the invention provides for a BCMA antigen binding protein for use in the treatment of cancer in a patient, wherein the patient expresses soluble BCMA in a sample from the patient.

In a further embodiment, the invention contemplates a pharmaceutical composition comprising a BCMA antigen binding protein and at least one pharmaceutically acceptable excipient for use in treating cancer in a patient, wherein the patient is characterized by a high level of soluble BCMA expression in a sample from the patient. In another embodiment, the invention provides a pharmaceutical composition comprising a BCMA antigen binding protein and at least one pharmaceutically acceptable excipient for use in treating cancer in a patient, wherein the patient expresses soluble BCMA in a sample from the patient.

The invention provides for a kit for the treatment of cancer with a BCMA antigen binding protein in a patient, comprising a means for determining the level of soluble BCMA in a sample from the patient.

The invention also provides for the use of a BCMA antigen binding protein in the manufacture of a medicament for the treatment of cancer in a patient, wherein a sample obtained from the patient is determined to express soluble BCMA. In another embodiment, the invention provides for the use of a BCMA antigen binding protein in the manufacture of a medicament for the treatment of cancer in a patient, wherein a sample obtained from the patient is determined to have a high level of soluble BCMA expression.

### BRIEF DESCRIPTION OF THE DRAWINGS

FIG. 1 demonstrates exemplary assays for detecting sBCMA. FIG. 1a demonstrates one exemplary method for the detection of free sBCMA (sBCMA not bound to a BCMA antigen binding protein). FIG. 1b demonstrates one exemplary method for the detection of bound sBCMA (sBCMA bound to J6M0-MMAF—a BCMA antigen binding protein).

FIG. 2 demonstrates levels of baseline soluble BCMA in healthy patients, multiple myeloma patients, and patients enrolled in a clinical study.

FIG. 3 demonstrates the best confirmed response obtained for each patient treated with a BCMA antigen binding protein relative to the baseline measures of sBCMA.

FIG. 4 demonstrates the reduction in free sBCMA relative to the dose level of an administered BCMA antigen binding protein.

### DETAILED DESCRIPTION OF THE INVENTION

The invention provides methods of diagnosing, for determining the prognosis of, or for optimizing treatment plans for, cancer patients by determining the presence or amount of soluble BCMA expression in a patient sample. It has been discovered that expression of soluble BCMA can be used as a biomarker to diagnose or determine the prognosis in cancer patients. Furthermore, it has been discovered that the presence or expression level of soluble BCMA in cancer patients can be used to select certain patient populations for treatment with a BCMA antigen binding protein and to inform dosing and treatment regimens by a clinician.

In one embodiment, the patient is a human patient or human subject.

Without being bound by theory, it is believed that sBCMA can bind to and inhibit the effects of therapeutic BCMA antigen binding proteins that are meant to target BCMA receptor bound to tumor cells.

### B-Cell Maturation Antigen (BCMA)

B-cell maturation antigen (“BCMA” or “TNFRSF17”) is a plasma cell expressed type-II transmembrane receptor that is a member of the tumour necrosis factor receptor superfamily (TNFRSF). It is responsible for driving the maturation of B-cells to long-lived plasma cells and is a potent activator of nuclear factor kappa light chain enhancer of activated B-cells (NFKB) NFKB are a family of transcription factors that are activated by pro-inflammatory cytokines or cell-bound ligands such as BCMA. Activation of this factor is associated with B-cell proliferation, survival, differentiation, and cellular apoptosis. (Bossen and Schneider, 2006). BCMA signalling has been implicated as a factor which supports the long-lived characteristics of malignant plasma cells. This has led to BCMA being a potential target for various plasma cell cancers, such as multiple myeloma.

Human BCMA contains the amino acid sequence of GenBank Accession Number Q02223.2 (SEQ ID NO: 11), or genes encoding human BCMA having at least 90 percent homology or at least 90 percent identity to SEQ ID NO: 11:

MLQMAGQCSQNEYFDSLLHACIPCQLRCSSNTPPLTCQRYCNASVTNSVK
GTNAILWTCLGLSLIISLAVFVLMFLLRKINSEPLKDEFKNTGSGLLGMA
NIDLEKSRTGDEIILPRGLEYTVEECTCEDCIKSKPKVDSDHCFPLPAME
EGATILVTTKTNDYCKSLPAALSATEIEKSISAR

### Soluble BCMA

BCMA can form a soluble or secreted form (“soluble BCMA” or “sBCMA”) (Rennert 2000). Without being bound by theory, it is believed that the extracellular portion of the BCMA receptor is cleaved from the membrane of the plasma cell surface via enzymes such as y-secretase (Laurent 2015). The soluble form of BCMA can easily be detected in human blood samples. As described herein, it has been discovered that sBCMA can be used as a biomarker for predicting patient outcome, determining prognosis, and optimizing treatment plans for cancer patients (e.g. B-cell cancers such as multiple myeloma and various lymphomas).

In one embodiment, the invention provides for soluble BCMA for use as a biomarker in a method of diagnosis comprising (a) obtaining a sample from the patient; and (b) testing the sample for the presence of soluble BCMA expression. In one embodiment, if the patient expresses soluble BCMA or expresses sBCMA at a high level, the patient is determined to have cancer. In another embodiment, a patient expresses a high level of BCMA when the amount of sBCMA is above about 10 ng/ml.

### Cancer

As used herein, the terms “cancer,” “neoplasm,” and “tumor” are used interchangeably and, in either the singular or plural form, refer to cells that have undergone a malignant transformation that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.” Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

In one aspect, the cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CIVIL), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

On one aspect, hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent B-cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphoma s(T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

In one aspect, hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenstrom's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

In one aspect the cancer is selected from head and neck cancer, breast cancer, lung cancer, colon cancer, ovarian cancer, prostate cancer, gliomas, glioblastoma, astrocytomas, glioblastoma multiforme, Bannayan-Zonana syndrome, Cowden disease, Lhermitte-Duclos disease, inflammatory breast cancer, Wilm's tumor, Ewing's sarcoma, Rhabdomyosarcoma, ependymoma, medulloblastoma, kidney cancer, liver cancer, melanoma, pancreatic cancer, sarcoma, osteosarcoma, giant cell tumor of bone, thyroid cancer, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), and testicular cancer.

In one aspect the human has a solid tumor. In one aspect the tumor is selected from head and neck cancer, gastric cancer, melanoma, renal cell carcinoma (RCC), esophageal cancer, non-small cell lung carcinoma, prostate cancer, colorectal cancer, ovarian cancer and pancreatic cancer. In another aspect the human has a liquid tumor such as diffuse large B cell lymphoma (DLBCL), multiple myeloma, chronic lyphomblastic leukemia (CLL), follicular lymphoma, acute myeloid leukemia and chronic myelogenous leukemia.

The present disclosure also relates to a method for treating or lessening the severity of a cancer selected from: brain (gliomas), glioblastomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, Hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

In one embodiment of the methods described herein, cancer includes multiple myeloma, lymphomas, chronic lymphocytic leukemia, non-Hodgkin's lymphoma, follicular lymphoma, and diffuse large B-cell lymphoma.

In one embodiment of the methods described herein, the cancer is multiple myeloma.

### Treatment and Prevention

By the term “treating” and grammatical variations thereof as used herein, is meant therapeutic therapy. In reference to a particular condition, treating means: (1) to ameliorate the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy is also contemplated herein. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

### Sample

Samples, e.g. biological samples, for testing or determining of levels of soluble BCMA may be any bodily fluid or tissue, including, but not limited to, serum, blood, blood components, urine, ascites fluid, bone marrow aspirate, and saliva. Testing for sBCMA levels may be conducted by several techniques known in the art and/or described herein. In some embodiments, the sample is serum.

### BCMA Antigen Binding Proteins

The BCMA antigen binding proteins described herein are useful in the treatment or prevention of cancers. “BCMA antigen binding protein” refers to any protein construct which is capable of binding to and/or neutralizing human BCMA.

The term “antigen binding protein” as used herein refers to proteins, protein fragments, antibodies, monoclonal antibodies, polyclonal antibodies, multi-specific antibodies (e.g. tri-specific and bispecific antibodies), antibody fragments, and other protein constructs which are capable of binding to human BCMA.

The term “monoclonal antibody” as used herein refers to an antibody obtained from a population of substantially homogenous antibodies i.e. the individual antibodies comprising the population are identical except for possible naturally occurring mutations that may be present in minor amounts. Monoclonal antibodies are highly specific being directed against a single antigenic binding site. Furthermore, in contrast to polyclonal antibody preparations which typically include different antibodies directed against different determinants (epitopes), each monoclonal antibody is directed against a single determinant on the antigen.

The antigen binding proteins of the present invention may comprise heavy chain variable regions and light chain variable regions of the invention which may be formatted into the structure of a natural antibody or functional fragment or equivalent thereof. An antigen binding protein of the invention may therefore comprise the VH regions of the invention formatted into a full length antibody, a (Fab′)2 fragment, a Fab fragment, or equivalent thereof (such as scFV, bi- tri- or tetra-bodies, Tandabs etc.), when paired with an appropriate light chain. The antibody may be an IgG1, IgG2, IgG3, or IgG4; or IgM; IgA, IgE or IgD or a modified variant thereof. The constant domain of the antibody heavy chain may be selected accordingly. The light chain constant domain may be a kappa or lambda constant domain. Furthermore, the antigen binding protein may comprise modifications of all classes, e.g. IgG dimers, Fc mutants that no longer bind Fc receptors or mediate C1q binding. The antigen binding protein may also be a chimeric antibody of the type described in WO86/01533 which comprises an antigen binding region and a non-immunoglobulin region.

The term “variant” as used herein refers to an amino acid sequence with at least one amino acid variation compared to the reference amino acid sequence and may include, for example, deletions, additions, insertions, translocations, truncations, and/or substitutions.

In one aspect of the invention, the antigen binding protein comprises a mAbdAb, dAbmAb, dAb, ScFv, Fab, Fab′, F(ab′)2, Fv, Fc, Fd, diabody, affibody, triabody, tetrabody, miniantibody, or a minibody.

In one embodiment, the BCMA antigen binding protein is a bispecific or trispecific antibody.

In one embodiment, the BCMA antigen protein is conjugated to a drug or cytotoxin. In another embodiment, the BCMA antigen binding protein is an antibody drug conjugate (ADC). In one aspect of the invention the BCMA antigen binding protein is a bi-specific T-cell engager (BiTE). In another embodiment, the BiTE comprises a fusion protein consisting of two single-chain variable fragments (scFvs) of different antibodies.

In one aspect of the invention, the BCMA antigen binding protein is a CAR-T (chimeric antigen receptor T-cell therapeutic). In a further aspect the CAR comprises a binding domain, a transmembrane domain and an intracellular effector domain. Chimeric antigen receptors (CARs) have been developed as artificial T cell receptors to generate novel specificities in T cells without the need to bind to MHC-antigenic peptide complexes. These synthetic receptors contain a target binding domain that is associated with one or more signaling domains via a flexible linker in a single fusion molecule. The target binding domain is used to target the T cell to specific targets on the surface of pathologic cells and the signaling domains contain molecular machinery for T cell activation and proliferation. The flexible linker which passes through the T cell membrane (i.e. forming a transmembrane domain) allows for cell membrane display of the target binding domain of the CAR. CARS have successfully allowed T cells to be redirected against antigens expressed at the surface of tumor cells from various malignancies including lymphomas and solid tumors.

In one aspect of the present invention the antigen binding protein is a humanized or chimeric antibody, in a further aspect the antibody is humanized. In one aspect the antibody is a monoclonal antibody.

A “chimeric antibody” refers to a type of engineered antibody in which a portion of the heavy and/or light chain is identical with or homologous to corresponding sequences in antibodies derived from a particular donor antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity. A suitable human acceptor antibody may be one selected from a conventional database, e.g. the KABATS database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody.

Exemplary BCMA antigen binding proteins and methods of making the same are disclosed in International Publication No. WO2012/163805 which is incorporated by reference herein in its entirety. Additional exemplary BCMA antigen binding proteins include those described in WO2016/014789, WO2016/090320, WO2016/090327, WO2016/020332, WO2016/079177, WO2014/122143, WO2014/122144, WO2017/021450, WO2016/014565, WO2014/068079, WO2015/166649, WO2015/158671, WO2015/052536, WO2014/140248, WO2013/072415, WO2013/072406, WO2014/089335, US2017/165373, WO2013/154760, and WO2017/051068, each of which is incorporated by reference herein in its entirety.

In one embodiment, the BCMA antigen binding protein has enhanced antibody dependent cell mediated cytotoxic activity (ADCC) effector function. The term “Effector Function” as used herein is meant to refer to one or more of Antibody dependent cell mediated cytotoxic activity (ADCC), Complement-dependent cytotoxic activity (CDC) mediated responses, Fc-mediated phagocytosis and antibody recycling via the FcRn receptor. For IgG antibodies, effector functionalities including ADCC and ADCP are mediated by the interaction of the heavy chain constant region with a family of Fcgamma receptors present on the surface of immune cells. In humans these include FcgammaRl (CD64), FcgammaRII (CD32) and FcgammaRIII (CD16). Interaction between the antigen binding protein bound to antigen and the formation of the Fc/Fcgamma complex induces a range of effects including cytotoxicity, immune cell activation, phagocytosis and release of inflammatory cytokines.

In another embodiment, the BCMA antigen binding proteins described herein inhibit the binding of BAFF and/or APRIL to the BCMA receptor. In another embodiment, the BCMA antigen binding proteins described herein are capable of binding to FcgammaRIIIA or is capable of FcgammaRIIIA mediated effector function.

“CDRs” are defined as the complementarity determining region amino acid sequences of an antibody which are the hypervariable domains of immunoglobulin heavy and light chains. There are three heavy chain and three light chain CDRs (or CDR regions) in the variable portion of an immunoglobulin. Thus, “CDRs” as used herein may refer to all three heavy chain CDRs, or all three light chain CDRs (or both all heavy and all light chain CDRs, if appropriate). CDRs provide the majority of contact residues for the binding of the antibody to the antigen or epitope. CDRs of interest in this invention are derived from donor antibody variable heavy and light chain sequences, and include analogs of the naturally occurring CDRs, which analogs also share or retain the same antigen binding specificity and/or neutralizing ability as the donor antibody from which they were derived. The CDR sequences of antibodies can be determined by the Kabat numbering system.

The terms “VH” and “VL” are used herein to refer to the heavy chain variable domain and light chain variable domain respectively of an antibody.

Exemplary BCMA antigen binding proteins are described in WO2012/163805, the disclosure of which is incorporated in its entirety herein.

In one embodiment, the BCMA antigen binding protein is an antibody comprising a heavy chain variable region CDR1 (“CDRH1”) comprising an amino acid sequence with at least 90%, 91%, 92%, 93%, 94%, 95%, 96%, 97%, 98%, 99% or 100% sequence identity to the amino acid sequence set forth in SEQ ID NO:1:

NYWMH

In one embodiment, the heavy chain variable region CDR1 (“CDRH1”) comprises an amino acid sequence with one amino acid variation (variant) to the amino acid sequence set forth in SEQ ID NO:1.

In one embodiment, the BCMA antigen binding protein is an antibody comprising a heavy chain variable region CDR2 (“CDRH2”) comprising an amino acid sequence with at least 90%, 91%, 92%, 93%, 94%, 95%, 96%, 97%, 98%, 99% or 100% sequence identity to the amino acid sequence set forth in SEQ ID NO:2:

ATYRGHSDTYYNQKFKG

In one embodiment, the heavy chain variable region CDR2 (“CDRH2”) comprises an amino acid sequence with one amino acid variation (variant) to the amino acid sequence set forth in SEQ ID NO:2.

In one embodiment, the BCMA antigen binding protein is an antibody comprising a heavy chain variable region CDR3 (“CDRH3”) comprising an amino acid sequence with at least 90%, 91%, 92%, 93%, 94%, 95%, 96%, 97%, 98%, 99% or 100% sequence identity to the amino acid sequence set forth in SEQ ID NO:3:

GAIYDGYDVLDN

In one embodiment, the heavy chain variable region CDR3 (“CDRH3”) comprises an amino acid sequence with one amino acid variation (variant) to the amino acid sequence set forth in SEQ ID NO:3.

In one embodiment, the BCMA antigen binding protein is an antibody comprising a light chain variable region CDR1 (“CDRL1”) comprising an amino acid sequence with at least 90%, 91%, 92%, 93%, 94%, 95%, 96%, 97%, 98%, 99% or 100% sequence identity to the amino acid sequence set forth in SEQ ID NO:4:

SASQDISNYLN

In one embodiment, the light chain variable region CDL1 (“CDR1”) comprises an amino acid sequence with one amino acid variation (variant) to the amino acid sequence set forth in SEQ ID NO:4.

In one embodiment, the BCMA antigen binding protein is an antibody comprising a light chain variable region CDR2 (“CDRL2”) comprising an amino acid sequence with at least 90%, 91%, 92%, 93%, 94%, 95%, 96%, 97%, 98%, 99% or 100% sequence identity to the amino acid sequence set forth in SEQ ID NO:5:

YTSNLHS

In one embodiment, the light chain variable region CDL2 (“CDR2”) comprises an amino acid sequence with one amino acid variation (variant) to the amino acid sequence set forth in SEQ ID NO:5.

In one embodiment, the BCMA antigen binding protein is an antibody comprising a light chain variable region CDR3 (“CDRL3”) comprising an amino acid sequence with at least 90%, 91%, 92%, 93%, 94%, 95%, 96%, 97%, 98%, 99% or 100% sequence identity to the amino acid sequence set forth in SEQ ID NO:6:

QQYRKLPWT

In one embodiment, the light chain variable region CDL3 (“CDR3”) comprises an amino acid sequence with one amino acid variation (variant) to the amino acid sequence set forth in SEQ ID NO:6.

In one embodiment, the BCMA antigen binding protein is an antibody comprising a CDRH1 comprising an amino acid sequence with at least 90%, 91%, 92%, 93%, 94%, 95%, 96%, 97%, 98%, 99% or 100% sequence identity to the amino acid sequence set forth in SEQ ID NO:1; CDRH2 comprising an amino acid sequence with at least 90%, 91%, 92%, 93%, 94%, 95%, 96%, 97%, 98%, 99% or 100% sequence identity to the amino acid sequence set forth in SEQ ID NO:2; CDRH3 comprising an amino acid sequence with at least 90%, 91%, 92%, 93%, 94%, 95%, 96%, 97%, 98%, 99% or 100% sequence identity to the amino acid sequence set forth in SEQ ID NO:3; CDRL1 comprising an amino acid sequence with at least 90%, 91%, 92%, 93%, 94%, 95%, 96%, 97%, 98%, 99% or 100% sequence identity to the amino acid sequence set forth in SEQ ID NO:4; CDRL2 comprising an amino acid sequence with at least 90%, 91%, 92%, 93%, 94%, 95%, 96%, 97%, 98%, 99% or 100% sequence identity to the amino acid sequence set forth in SEQ ID NO: 5; and/or CDRL3 comprising an amino acid sequence with at least 90%, 91%, 92%, 93%, 94%, 95%, 96%, 97%, 98%, 99% or 100% sequence identity to the amino acid sequence set forth in SEQ ID NO:6.

In one embodiment, the BCMA antigen binding protein is an antibody comprising a CDRH1 comprising an amino acid sequence set forth in SEQ ID NO:1; a CDRH2 comprising an amino acid sequence set forth in SEQ ID NO:2; a CDRH3 comprising an amino acid sequence set forth in SEQ ID NO:3; a CDRL1 comprising an amino acid sequence set forth in SEQ ID NO:4; a CDRL2 comprising an amino acid sequence set forth in SEQ ID NO:5; and a CDRL3 comprising an amino acid sequence set forth in SEQ ID NO:6.

In one embodiment, the BCMA antigen binding protein is an antibody comprising a heavy chain variable region (“VH”) comprising an amino acid sequence with at least 90%, 91%, 92%, 93%, 94%, 95%, 96%, 97%, 98%, 99% or 100% sequence identity to the amino acid sequence set forth in SEQ ID NO:7:

QVQLVQSGAEVKKPGSSVKVSCKASGGTFSNYWMHWVRQAPGQGLEWM
GATYRGHSDTYYNQKFKGRVTITADKSTSTAYMELSSLRSEDTAVYYCAR
GAIYDGYDVLDNVVGQGTLVTVSS

In one embodiment, the BCMA antigen binding protein is an antibody comprising a light chain variable region (“VL”) comprising an amino acid sequence with at least 90%, 91%, 92%, 93%, 94%, 95%, 96%, 97%, 98%, 99% or 100% sequence identity to the amino acid sequence set forth in SEQ ID NO:8:

DIQMTQSPSSLSASVGDRVTITCSASQDISNYLNWYQQKPGKAPKLLIYY
TSNLHSGVPSRFSGSGSGTDFTLTISSLQPEDFATYYCQQYRKLPWTFGQ
GTKLEIKR

In one embodiment, the BCMA antigen binding protein is an antibody comprising a VH comprising an amino acid sequence with at least 90%, 91%, 92%, 93%, 94%, 95%, 96%, 97%, 98%, 99% or 100% sequence identity to the amino acid sequence set forth in SEQ ID NO:7; and a VL comprising an amino acid sequence with at least 90%, 91%, 92%, 93%, 94%, 95%, 96%, 97%, 98%, 99% or 100% sequence identity to the amino acid sequence set forth in SEQ ID NO:8.

In one embodiment, the BCMA antigen binding protein is an antibody comprising a VH comprising an amino acid sequence set forth in SEQ ID NO:7; and a VL comprising an amino acid sequence set forth in SEQ ID NO:8. (herein referred to as “J6M0”).

In one embodiment, the BCMA antigen binding protein is an antibody comprising a heavy chain region (“HC”) comprising an amino acid sequence with at least 90%, 91%, 92%, 93%, 94%, 95%, 96%, 97%, 98%, 99% or 100% sequence identity to the amino acid sequence set forth in SEQ ID NO:9:

QVQLVQSGAEVKKPGSSVKVSCKASGGTFSNYWMHWVRQAPGQGLEW
MGATYRGHSDTYYNQKFKGRVTITADKSTSTAYMELSSLRSEDTAVYYC
ARGAIYDGYDVLDNWGQGTLVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLS
LSPGK

In one embodiment, the BCMA antigen binding protein is an antibody comprising a light chain region (“LC”) comprising an amino acid sequence with at least 90%, 91%, 92%, 93%, 94%, 95%, 96%, 97%, 98%, 99% or 100% sequence identity to the amino acid sequence set forth in SEQ ID NO:10:

DIQMTQSPSSLSASVGDRVTITCSASQDISNYLNWYQQKPGKAPKLLIYY
TSNLHSGVPSRFSGSGSGTDFTLTISSLQPEDFATYYCQQYRKLPWTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

In one embodiment, the BCMA antigen binding protein is an antibody comprising a HC comprising an amino acid sequence with at least 90%, 91%, 92%, 93%, 94%, 95%, 96%, 97%, 98%, 99% or 100% sequence identity to the amino acid sequence set forth in SEQ ID NO:9; and a LC comprising an amino acid sequence with at least 90%, 91%, 92%, 93%, 94%, 95%, 96%, 97%, 98%, 99% or 100% sequence identity to the amino acid sequence set forth in SEQ ID NO:10.

In one embodiment, the BCMA antigen binding protein is an antibody comprising a HC comprising an amino acid sequence set forth in SEQ ID NO:9; and a LC comprising an amino acid sequence set forth in SEQ ID NO:10.

In one embodiment, the BCMA antigen binding protein is conjugated to a drug or cytotoxin. In another embodiment, the BCMA antigen binding protein is an antibody-drug-conjugate (ADC or an immunoconjugate). The ADC may comprise any BCMA antigen binding protein described herein conjugated to one or more cytotoxic agents, such as a chemotherapeutic agent, a drug, a growth inhibitory agent, a toxin (e.g., a protein toxin, an enzymatically active toxin of bacterial, fungal, plant, or animal origin, or fragments thereof), or a radioactive isotope (i.e., a radioconjugate).

In one embodiment, the BCMA antigen binding protein is an immunoconjugate having the following general structure:

ABP-((Linker)n-Ctx)m

wherein

ABP is an antigen binding protein

Linker is either absent or any a cleavable or non-cleavable linker

Ctx is any cytotoxic agent described herein

n is 0, 1, 2, or 3 and

m is 1, 2, 3, 4, 5, 6, 7, 8, 9 or 10.

Exemplary cytotoxic agents include auristatins (e.g., monomethyl auristatin E (MMAE) or monomethyl auristatin F (MMAF)); sequence-selective DNA minor-groove binding crosslinking agents (e.g., pyrrolobenzodiazepine (PBD)); maytansinoids (e.g. DM1 or DM4); and alpha-amanitin cyclic peptides.

Exemplary linkers include protease cleavable linkers, 6-maleimidocaproyl (MC), maleimidopropanoyl (MP), valine-citrulline (val-cit), alanine-phenylalanine (ala-phe), p-aminobenzyloxycarbonyl (PAB), N-Succinimidyl 4-(2-pyridylthio)pentanoate (SPP), N-succinimidyl 4-(N-maleimidomethyl)cyclohexane-1 carboxylate (SMCC), 4-(N-maleimidomethyl) cyclohexane-1-carboxylate (MCC), and N-Succinimidyl (4-iodo-acetyl) aminobenzoate (SIAB).

In one embodiment, the BCMA antigen binding protein is an immunoconjugate containing a monoclonal antibody linked to MMAE or MMAF. In another embodiment, the BCMA antigen binding protein is an immunoconjugate containing a monoclonal antibody linked to MMAE or MMAF by an MC linker as depicted in the following structures:

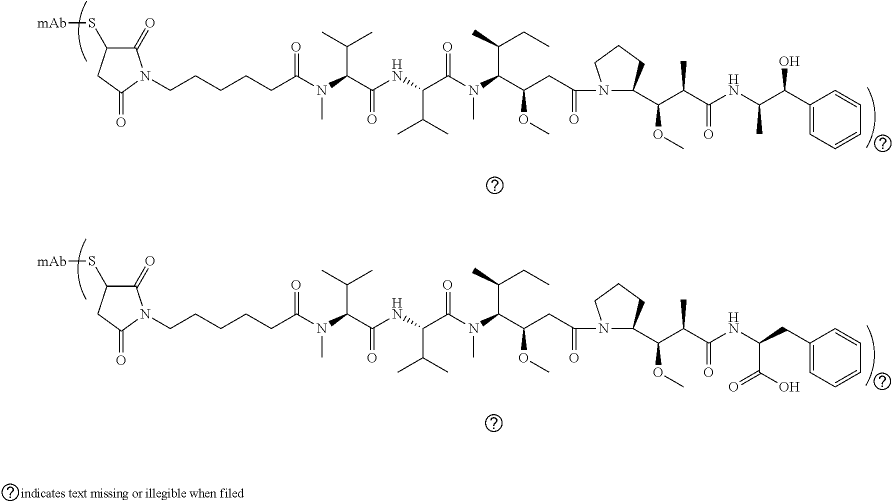

In one embodiment, the BCMA antigen binding protein is a monoclonal antibody comprising a VH comprising an amino acid sequence set forth in SEQ ID NO:7; and a VL comprising an amino acid sequence set forth in SEQ ID NO:8, wherein the antibody is conjugated to MMAF (herein referred to as “J6M0-MMAF”).

Exemplary CAR-T therapeutics include Bb2121 or Bb2127 (Celgene/Bluebird), JCARH125 or FCARH143 (Celgene/Juno), LCAR-B38M (Nanjing/Janssen/Genscript), MCARH171/ET140 (Celgene/Juno/Eureka), DESCARTES-08 (Cartesian), KITE-585 (Gilead/Kite), and P-BCMA-101 (Poseida).

Exemplary monoclonal antibodies, bispecific antibodies, trispecific antibodies, duobodies, or BiTes include CC-93269/EM801 (Celgene/EngMab), AMG 701 or AMG 420 (Amgen), JNJ-64007957 (Janssen), SEA-BCMA (Seattle Genetics), and PF-06863135 (Pfizer).

Exemplary ADCs include MEDI2228 (Medimmune), AMG 224 (Amgen), and HDP-101 (Heidelberg Max Eder).

The appropriate therapeutically effective dose of the BCMA antigen binding protein will be determined readily by those of skill in the art. As used herein, the term “effective dose” means that dose of a drug or pharmaceutical agent that will elicit the biological or medical response of a tissue, system, animal or human that is being sought, for instance, by a researcher or clinician. Furthermore, the term “therapeutically effective dose” means any dose which, as compared to a corresponding subject who has not received such dose, results in improved treatment, healing, prevention, or amelioration of a disease, disorder, or side effect, or a decrease in the rate of advancement of a disease or disorder. The term also includes within its scope doses effective to enhance normal physiological function.

Suitable doses of the BCMA antigen binding proteins described herein may be calculated for patients according to their weight, for example suitable doses may be in the range of about 0.1 to about 20 mg/kg, for example about 1 to about 20 mg/kg, for example about 10 to about 20 mg/kg or for example about 1 to about 15 mg/kg, for example about 10 to about 15 mg/kg.

In one embodiment, the therapeutically effective dose of the BCMA antigen binding protein is in the range of about 0.03 mg/kg to about 4.6 mg/kg. In yet another embodiment, the therapeutically effective dose of the BCMA antigen binding protein is 0.03 mg/kg, 0.06 mg/kg, 0.12 mg/kg, 0.24 mg/kg, 0.48 mg/kg, 0.96 mg/kg, 1.92 mg/kg, 2.5 mg/kg, 3.4 mg/kg, or 4.6 mg/kg. In yet another embodiment, the therapeutically effective dose of the anti-BCMA antigen binding protein is 1.9 mg/kg, 2.5 mg/kg or 3.4 mg/kg.

One aspect of the invention provides for a BCMA antigen binding protein for use in the treatment of cancer in a patient, wherein the patient is characterized by expressing soluble BCMA in a sample from the patient.

One aspect of the invention provides for a BCMA antigen binding protein for use in the treatment of cancer in a patient, wherein the patient is characterized by a high level of soluble BCMA expression in a sample from the patient.

In another aspect, the invention provides for a BCMA antigen binding protein for use in treating patients identified to have a high level of soluble BCMA. In another aspect, the invention provides for a BCMA antigen binding protein for use in treating patients identified to have a high level of soluble BCMA; the level of sBCMA is above 10 ng/ml and wherein the BCMA antigen binding protein is at a concentration per human dose/in an amount of at least about 1.92 mg/kg.

### Pharmaceutical Compositions

One aspect of the invention provides for a pharmaceutical composition comprising a BCMA antigen binding protein and at least one pharmaceutically acceptable excipient for use in the treatment of cancer in a patient, wherein the patient is characterized by expressing soluble BCMA in a sample from the patient.

One aspect of the invention provides for a pharmaceutical composition comprising a BCMA antigen binding protein at least one pharmaceutically acceptable excipient for use in the treatment of cancer in a patient, wherein the patient is characterized by a high level of soluble BCMA expression in a sample from the patient.

### Methods and Uses

The invention provides for a method of diagnosing cancer in a patient, comprising: (a) obtaining a sample from the patient; and (b) testing the sample for the presence of soluble BCMA expression. In one embodiment, if the patient expresses soluble BCMA or expresses sBCMA at a high level, the patient is determined to have cancer.

In another embodiment, a method of diagnosing cancer in a patient, comprises: (a) obtaining a sample from the patient; and (b) testing the sample for the presence of soluble BCMA expression, wherein if the patient expresses sBCMA at a high level, the patient is determined to have cancer, wherein a high level of sBCMA is above about 5 ng/ml, above about 10 ng/ml, above about 20 ng/ml, above about 30 ng/ml, above about 40 ng/ml, above about 50 ng/ml, above about 60 ng/ml, above about 70 ng/ml, above about 80 ng/ml, above about 90 ng/ml, above about 100 ng/ml, above about 200 ng/ml, above about 300 ng/ml, above about 400 ng/ml, above about 500 ng/ml, above about 600 ng/ml, above about 700 ng/ml, above about 800 ng/ml, or above about 900 ng/ml.

The invention further provides for methods of determining the prognosis of cancer in a patient (e.g. a human subject), comprising (a) obtaining a sample from a patient; and (b) testing the sample for the level of soluble BCMA expression. In one embodiment, if the patient expresses soluble BCMA, then the prognosis, or outcome, is poor. In another embodiment, if the patient expresses a high level of soluble BCMA, then the prognosis, or outcome, is poor.

Cancer prognosis is often measured using survival rates. Cancer statistics often use an overall five-year survival rate. Disease-free survival rate is the number of people who have no evidence of cancer after treatment. Progression-free survival rate is the number of people who have been treated for cancer and either have no signs of cancer recurrence or who have cancer that has remained stable without growing. In another embodiment, prognosis is poor when the patient has less than less than about 70%, about 60%, less than about 50%, less than about 40%, less than about 30%, less than about 20%, less than about 15%, less than about 10%, or less than about 5% chance of survival using cancer prognosis statistical methods know to those skilled in the art.

In one embodiment, the patient's prognosis is poor when the patient expresses any amount of sBCMA. In one embodiment, the patient's prognosis is poor when the patient expresses any amount of sBCMA compared to a reference sample.

In one embodiment, the patient's prognosis is poor when the patient expresses a high level of soluble BCMA, wherein a high level of sBCMA is above about 5 ng/ml, above about 10 ng/ml, above about 20 ng/ml, above about 30 ng/ml, above about 40 ng/ml, above about 50 ng/ml, above about 60 ng/ml, above about 70 ng/ml, above about 80 ng/ml, above about 90 ng/ml, above about 100 ng/ml, above about 200 ng/ml, above about 300 ng/ml, above about 400 ng/ml, above about 500 ng/ml, above about 600 ng/ml, above about 700 ng/ml, above about 800 ng/ml, or above about 900 ng/ml.

In another embodiment, the patient's prognosis is poor when the patient expresses greater than about 10 ng/mL soluble BCMA. In another embodiment, the patient's prognosis of multiple myeloma is poor when the patient expresses greater than about long/mL soluble BCMA.

One aspect of the invention provides for methods of predicting a patient's response to treatment with a BCMA antigen binding protein comprising: (a) obtaining a sample from a patient; and (b) testing the sample for the level of soluble BCMA expression, wherein if the patient expresses soluble BCMA, the patient is predicted to not respond to treatment with a BCMA antigen binding protein.

One aspect of the invention provides for methods of predicting a patient's response to treatment with a BCMA antigen binding protein comprising: (a) obtaining a sample from a patient; and (b) testing the sample for the level of soluble BCMA expression, wherein if the patient expresses a high level of soluble BCMA, the patient is predicted to not respond to treatment with a BCMA antigen binding protein.

The term “response” is known to those skilled in the art. Guidance documents in particular cancer fields are known to those skilled in the art and provide definitions for “response” for a given cancer type. For example, “response” may include stringent complete remission (sCR), complete remission (CR), near complete remission (nCR), very good partial response (VGPR), partial response (PR), and/or stable disease (SD). Exemplary guidance for defining response to treatment in multiple myeloma patients is provided by the Center for International Blood & Marrow transplant Research group (CIBMTR) (See https://www.cibmtr.org/manuals/fim/1/en/topic/multiple-myeloma-response-criteria, 2018, the disclosure of which is incorporated herein in its entirety). In one embodiment, response to treatment in multiple myeloma patients is defined as stringent complete remission (sCR), complete remission (CR), near complete remission (nCR), very good partial response (VGPR), or partial response (PR).

In another aspect methods of predicting a multiple myeloma patient's response to treatment with a BCMA antigen binding protein comprise: (a) obtaining a sample from a patient; and (b) testing the sample for the level of soluble BCMA expression, wherein if the patient expresses less than about 100 ng/ml, less than about 90 ng/ml, less than about 80 ng/ml, less than about 70 ng/ml, less than about 60 ng/ml, less than about 50 ng/ml, less than about 40 ng/ml, less than about 30 ng/ml, less than about 20 ng/ml, less than about 10 ng/ml, or less than about 5 ng/ml, of soluble BCMA, the patient is predicted to respond to treatment with a BCMA antigen binding protein.

In another aspect methods of predicting a multiple myeloma patient's response to treatment with a BCMA antigen binding protein comprise: (a) obtaining a sample from a patient; and (b) testing the sample for the level of soluble BCMA expression, wherein if the patient expresses less than about 50 ng/ml of soluble BCMA, the patient is predicted to respond to treatment with a BCMA antigen binding protein.

In another aspect methods of predicting a multiple myeloma patient's response to treatment with a BCMA antigen binding protein comprise: (a) obtaining a sample from a patient; and (b) testing the sample for the level of soluble BCMA expression, wherein if the patient expresses greater than about 1 Ong/ml, greater than about 20 ng/ml, greater than about 30 ng/ml, greater than about 40 ng/ml, greater than about 50 ng/ml, greater than about 60 ng/ml, greater than about 70 ng/ml, greater than about 80 ng/ml, greater than about 90 ng/ml, or greater than about 100 ng/ml of soluble BCMA, the patient is predicted to not respond to treatment with a BCMA antigen binding protein.

In another aspect methods of predicting a multiple myeloma patient's response to treatment with a BCMA antigen binding protein comprise: (a) obtaining a sample from a patient; and (b) testing the sample for the level of soluble BCMA expression, wherein if the patient expresses greater than about 50 ng/ml of soluble BCMA, the patient is predicted to not respond to treatment with a BCMA antigen binding protein.

In another aspect methods of predicting a multiple myeloma patient's response to treatment with a BCMA antigen binding protein comprise: (a) obtaining a sample from a patient; and (b) testing the sample for the level of soluble BCMA expression, wherein if the patient expresses greater than about 40 ng/ml of soluble BCMA, the patient is predicted to not respond to treatment with a BCMA antigen binding protein.

In another aspect methods of predicting a multiple myeloma patient's response to treatment with a BCMA antigen binding protein comprise: (a) obtaining a sample from a patient; and (b) testing the sample for the level of soluble BCMA expression, wherein if the patient expresses greater than about 50 ng/ml of soluble BCMA, the patient is predicted to not respond to treatment with a BCMA antigen binding protein.

In another aspect methods of predicting a multiple myeloma patient's response to treatment with a BCMA antigen binding protein comprise: (a) obtaining a sample from a patient; and (b) testing the sample for the level of soluble BCMA expression, wherein if the patient expresses greater than about 40 ng/ml of soluble BCMA, the patient is predicted to not respond to treatment with a BCMA antigen binding protein comprising a VH comprising an amino acid sequence set forth in SEQ ID NO:7; a VL comprising an amino acid sequence set forth in SEQ ID NO:8, and wherein the antibody is conjugated to MMAF.

The invention provides for methods of treating cancer in a patient in need thereof, comprising: (a) determining if the patient expresses soluble BCMA in a sample obtained from the patient, and (b) if the patient expresses soluble BCMA, administering to the patient an effective amount of a BCMA antigen binding protein.

In one embodiment, the invention provides for a method for treating cancer in a patient in need thereof, comprising determining if a patient expresses soluble BCMA, wherein if the patient is determined to express soluble BCMA, administering to the patient an effective amount of a BCMA antigen binding protein.

In one embodiment, the invention provides for a method for treating cancer in a patient in need thereof, comprising determining if a patient expresses a high level of soluble BCMA, wherein if the patient is determined to express a high level of soluble BCMA, administering to the patient an effective amount of a BCMA antigen binding protein.

The invention provides for methods of treating cancer in a patient in need thereof, comprising: (a) determining if the patient expresses soluble BCMA in a sample obtained from the patient, and (b) if the patient expresses soluble BCMA, administering to the patient an effective amount of a BCMA antigen binding protein, wherein the patient is determined to have multiple myeloma, lymphoma, chronic lymphocytic leukemia, non-Hodgkin's lymphoma, follicular lymphoma, or diffuse large B-cell lymphoma, and wherein the antigen binding comprises a VH comprising an amino acid sequence set forth in SEQ ID NO:7; a VL comprising an amino acid sequence set forth in SEQ ID NO:8, and wherein the antibody is conjugated to MMAF.

The invention provides for methods of treating cancer in a patient in need thereof, comprising: (a) determining if the patient expresses a high level soluble BCMA in a sample obtained from the patient, and (b) if the patient expresses a high level of soluble BCMA, administering to the patient an effective amount of a BCMA antigen binding protein. The invention provides for methods of treating cancer in a patient in need thereof, comprising: (a) determining if the patient expresses soluble BCMA in a sample obtained from the patient, and (b) if the patient expresses a high level of soluble BCMA, administering to the patient an effective amount of a BCMA antigen binding protein, wherein the patient is determined to have multiple myeloma, lymphoma, chronic lymphocytic leukemia, non-Hodgkin's lymphoma, follicular lymphoma, or diffuse large B-cell lymphoma, wherein the antigen binding comprises a VH comprising an amino acid sequence set forth in SEQ ID NO:7; a VL comprising an amino acid sequence set forth in SEQ ID NO:8, and wherein the antibody is conjugated to MMAF, and wherein the sBCMA expression is high when the level of sBMCA is at least about 10 ng/ml.

The invention provides for methods of treating cancer in a patient in need thereof, comprising: (a) determining the level of soluble BCMA in a sample obtained from the patient, and (b) if the level of soluble BCMA is high, administering to the patient an effective amount of a BCMA antigen binding protein. In one embodiment, the level of sBCMA is high

The invention further provides for methods for treating cancer in a patient (e.g. human patient or subject) comprising: (a) obtaining a sample from the patient; (b) testing the sample for expression of soluble BCMA; and (c) if the patient expresses soluble BCMA, or expresses a high level of soluble BCMA, administering to the patient an effective amount of a BCMA antigen binding protein.

In one embodiment, the patient expresses soluble BCMA when the patient expresses any amount of sBCMA in the sample. In one embodiment, the patient expresses soluble BCMA when the patient expresses any amount of sBCMA in the sample compared to a reference sample.

In another embodiment, the invention further provides for methods for treating cancer in a patient (e.g. human patient or subject) comprising: (a) obtaining a sample from the patient; (b) testing the sample for expression of soluble BCMA; and (c) if the patient expresses a high level of soluble BCMA, administering to the patient an effective amount of a BCMA antigen binding protein, wherein the patient expresses a high level of soluble BCMA when the amount of sBCMA in the sample is above about 5 ng/ml, above about 10 ng/ml, above about 20 ng/ml, above about 30 ng/ml, above about 40 ng/ml, above about 50 ng/ml, above about 60 ng/ml, above about 70 ng/ml, above about 80 ng/ml, above about 90 ng/ml, above about 100 ng/ml, above about 200 ng/ml, above about 300 ng/ml, above about 400 ng/ml, above about 500 ng/ml, above about 600 ng/ml, above about 700 ng/ml, above about 800 ng/ml, or above about 900 ng/ml.

In another embodiment, the invention further provides for methods for treating cancer in a patient (e.g. human patient or subject) comprising: (a) obtaining a sample from the patient; (b) testing the sample for expression of soluble BCMA; and (c) if the patient expresses a high level of soluble BCMA, administering to the patient an effective amount of a BCMA antigen binding protein, wherein the patient expresses a high level of soluble BCMA when the amount of sBCMA in the sample is above about 10 ng/ml.

In another embodiment, the invention further provides for methods for treating cancer in a patient (e.g. human patient or subject) comprising: (a) obtaining a sample from the patient; (b) testing the sample for expression of soluble BCMA; and (c) if the patient expresses a high level of soluble BCMA, administering to the patient an effective amount of a BCMA antigen binding protein, wherein the patient expresses a high level of soluble BCMA when the amount of sBCMA in the sample is above about 10 ng/ml; wherein the cancer is selected from multiple myeloma, lymphoma, chronic lymphocytic leukemia, non-Hodgkin's lymphoma, follicular lymphoma, and diffuse large B-cell lymphoma.

In another embodiment, the invention further provides for methods for treating cancer in a patient (e.g. human patient or subject) comprising: (a) obtaining a sample from the patient; (b) testing the sample for expression of soluble BCMA; and (c) if the patient expresses a high level of soluble BCMA, administering to the patient an effective amount of a BCMA antigen binding protein, wherein the patient expresses a high level of soluble BCMA when the amount of sBCMA in the sample is above about 10 ng/ml; wherein the cancer is selected from multiple myeloma, lymphoma, chronic lymphocytic leukemia, non-Hodgkin's lymphoma, follicular lymphoma, and diffuse large B-cell lymphoma; and wherein the antigen binding comprises a VH comprising an amino acid sequence set forth in SEQ ID NO:7; a VL comprising an amino acid sequence set forth in SEQ ID NO:8, and wherein the antibody is conjugated to MMAF.

The invention provides for methods of selecting the dose of a BCMA antigen binding protein for treating cancer in a patient in need thereof, comprising: (a) obtaining a sample from the patient; (b) testing the sample for the level of soluble BCMA expression; and (c) if the patient has low soluble BCMA expression, treating the patient with a low dose of a BCMA antigen binding protein; or if the patient has high soluble BCMA expression, treating the patient with a high dose of a BCMA antigen binding protein.

In one embodiment, the invention provides for methods of selecting the dose of a BCMA antigen binding protein for treating cancer in a patient in need thereof, comprising: (a) obtaining a sample from the patient; (b) testing the sample for the level of soluble BCMA expression; and (c) if the patient has low soluble BCMA expression, treating the patient with a low dose of a BCMA antigen binding protein, wherein a patient has low soluble BCMA expression when the amount of soluble BCMA in the sample is below about 300 ng/ml, below about 200 ng/ml, below about 100 ng/ml, below about 90 ng/ml, below about 80 ng/ml, below about 70 ng/ml, below about 60 ng/ml, below about 50 ng/ml, below about 40 ng/ml, below about 30 ng/ml, below about 20 ng/ml, below about 10 ng/ml, or below about 5 ng/ml.

In one embodiment, the invention provides for methods of selecting the dose of a BCMA antigen binding protein for treating cancer in a patient in need thereof, comprising: (a) obtaining a sample from the patient; (b) testing the sample for the level of soluble BCMA expression; and (c) if the patient has low soluble BCMA expression, treating the patient with a low dose of a BCMA antigen binding protein, wherein a patient has low soluble BCMA expression when the amount of soluble BCMA in the sample is below about 10 ng/ml.

In one embodiment, the invention provides for methods of selecting the dose of a BCMA antigen binding protein for treating cancer in a patient in need thereof, comprising: (a) obtaining a sample from the patient; (b) testing the sample for the level of soluble BCMA expression; and (c) if the patient has high soluble BCMA expression, treating the patient with a high dose of a BCMA antigen binding protein, wherein a patient has high soluble BCMA expression when the amount of soluble BCMA in the sample is above about 5 ng/ml, above about 10 ng/ml, above about 20 ng/ml, above about 30 ng/ml, above about 40 ng/ml, above about 50 ng/ml, above about 60 ng/ml, above about 70 ng/ml, above about 80 ng/ml, above about 90 ng/ml, above about 100 ng/ml, above about 200 ng/ml, above about 300 ng/ml, above about 400 ng/ml, above about 500 ng/ml, above about 600 ng/ml, above about 700 ng/ml, above about 800 ng/ml, or above about 900 ng/ml.

In one embodiment, the invention provides for methods of selecting the dose of a BCMA antigen binding protein for treating cancer in a patient in need thereof, comprising: (a) obtaining a sample from the patient; (b) testing the sample for the level of soluble BCMA expression; and (c) if the patient has high soluble BCMA expression, treating the patient with a high dose of a BCMA antigen binding protein, wherein a patient has high soluble BCMA expression when the amount of soluble BCMA in the sample is above about 10 ng/ml.

In one embodiment a low dose of the BCMA antigen binding protein is less than 4.6 mg/kg. In another embodiment, a low dose of the BCMA antigen binding protein is 0.03 mg/kg, 0.06 mg/kg, 0.12 mg/kg, 0.24 mg/kg, 0.48 mg/kg, 0.96 mg/kg, 1.92 mg/kg, 2.5 mg/kg, or 3.4 mg/kg.

In one embodiment, a high dose of the BCMA antigen binding protein is greater than 0.96 mg/kg. In another embodiment, a high dose of the BCMA antigen binding protein is 0.96 mg/kg, 1.92 mg/kg, 2.5 mg/kg, 3.4 mg/kg, or 4.6 mg/kg.

One aspect of the invention provides for the use of a BCMA antigen binding protein in the manufacture of a medicament for the treatment of cancer in a patient, wherein a sample obtained from the patient subject is determined to express soluble BCMA. Another aspect of the invention provides for the use of a BCMA antigen binding protein in the manufacture of a medicament for the treatment of cancer in a patient, wherein a sample obtained from the patient is determined to have a high level of soluble BCMA expression.

In one embodiment, the patient expresses soluble BCMA when the patient expresses any amount of sBCMA in the sample. In another embodiment, the patient expresses a high level of soluble BCMA when the amount of sBCMA in the sample is above about 5 ng/ml, above about 10 ng/ml, above about 20 ng/ml, above about 30 ng/ml, above about 40 ng/ml, above about 50 ng/ml, above about 60 ng/ml, above about 70 ng/ml, above about 80 ng/ml, above about 90 ng/ml, above about 100 ng/ml, above about 200 ng/ml, above about 300 ng/ml, above about 400 ng/ml, above about 500 ng/ml, above about 600 ng/ml, above about 700 ng/ml, above about 800 ng/ml, or above about 900 ng/ml.

One aspect of the invention provides for the use of a BCMA antigen binding protein in the manufacture of a medicament for the treatment of cancer in a patient, wherein a sample obtained from the patient subject is determined to express soluble BCMA; and wherein the patient is determined to have multiple myeloma, lymphoma, chronic lymphocytic leukemia, non-Hodgkin's lymphoma, follicular lymphoma, and diffuse large B-cell lymphoma; and wherein the patients expresses at least about 10 ng/ml of sBCMA.

In any of the embodiments described herein, a patient is expresses a high level of soluble BCMA when the patient expresses soluble BCMA at a level above about 5 ng/ml, above about 10 ng/ml, above about 20 ng/ml, above about 30 ng/ml, above about 40 ng/ml, above about 50 ng/ml, above about 60 ng/ml, above about 70 ng/ml, above about 80 ng/ml, above about 90 ng/ml, above about 100 ng/ml, above about 200 ng/ml, above about 300 ng/ml, above about 400 ng/ml, above about 500 ng/ml, above about 600 ng/ml, above about 700 ng/ml, above about 800 ng/ml, or above about 900 ng/ml.

In some embodiments, in addition to treatment with the BCMA antigen binding protein, the patient can be further treated with one or more additional cancer therapeutics or anti-neoplastic agents. Typically, any anti-neoplastic agent that has activity versus a susceptible tumor being treated may be co-administered in the treatment of cancer in the present invention. A person of ordinary skill in the art would be able to discern which combinations of agents would be useful based on the particular characteristics of the drugs and the cancer involved.

Typical anti-neoplastic agents useful in the present invention include, but are not limited to, anti-microtubule or anti-mitotic agents such as diterpenoids and vinca alkaloids; platinum coordination complexes; thalidomide analogs (IMiDs); immunotherapeutic antibodies (e.g. anti-PD1, anti-PDL1, anti-CD38, anti-ICOS, anti-OX40); alkylating agents such as nitrogen mustards, oxazaphosphorines, alkylsulfonates, nitrosoureas, and triazenes; antibiotic agents such as actinomycins, anthracyclins, and bleomycins; topoisomerase I inhibitors such as camptothecins; topoisomerase II inhibitors such as epipodophyllotoxins; antimetabolites such as purine and pyrimidine analogues and anti-folate compounds; hormones and hormonal analogues; signal transduction pathway inhibitors; non-receptor tyrosine kinase angiogenesis inhibitors; immunotherapeutic agents; proapoptotic agents; cell cycle signalling inhibitors; proteasome inhibitors; heat shock protein inhibitors; inhibitors of cancer metabolism; chemotherapeutic agents; steroid (e.g. dexamethasone); immuno-modulatory agents; immuno-modulators; and immunostimulatory adjuvants.

In one embodiment, the additional anti-neoplastic agent is at least one selected from an anti-PD1 antibody, an anti-ICOS antibody, and anti-OX40 antibody, an anti-CD38 antibody, a proteasome inhibitor, a thalidomide analog, and dexamethasone.

The appropriate therapeutically effective dose of the additional cancer therapeutic or anti-neoplastic agent will be determined readily by those of skill in the art. As used herein, the term “effective dose” of the additional cancer therapeutic or anti-neoplastic agent means that dose of a drug or pharmaceutical agent that will elicit the biological or medical response of a tissue, system, animal or human that is being sought, for instance, by a researcher or clinician. Furthermore, the term “therapeutically effective dose” of the additional cancer therapeutic or anti-neoplastic agent means any dose which, as compared to a corresponding subject who has not received such dose, results in improved treatment, healing, prevention, or amelioration of a disease, disorder, or side effect, or a decrease in the rate of advancement of a disease or disorder. The term also includes within its scope doses effective to enhance normal physiological function.

### Measuring Soluble BCMA

Soluble BCMA in a sample can be measured by various techniques known in the art. Assays can be specific for detecting free circulating soluble BCMA as well as soluble BCMA bound to a BCMA antigen binding protein. For example, sBCMA levels in a sample can be measured using enzyme-linked immunosorbent assays (ELISA), Western blot assays, mass spectrometry, meso scale discovery (MSD), immunohistochemistry (IHC), immunoprecipitation, immunofluorescence, flow cytometry, or other antibody-based capture/detection methods. Various detection moieties may include biotin/streptavidin binding, colorimetric, ultra-violet, fluorescent, electrochemical, or other detection methods known to one skilled in the art.

Determining sBCMA presence or expression level can be accomplished by comparing the sample of interest to a reference sample or control sample. For example, the reference sample or control sample can be: 1) a sample known to contain no sBCMA (e.g. buffer control or sample from a healthy donor); 2) a negative control sample in which a particular assay reagent is purposely not included so as to obtain a negative signal; 3) several samples containing varying amounts of sBCMA to be used a standard curve for quantitating the amount of sBCMA in the sample of interest; or 4) other control or reference samples which are common practice to those skilled in the art.

In one embodiment, sBCMA is detected in a sample using means for detection that comprise a capture antibody and/or a detection antibody that binds to sBCMA (free or bound to BCMA antigen binding protein) in the sample, wherein the means comprise a detection moiety.

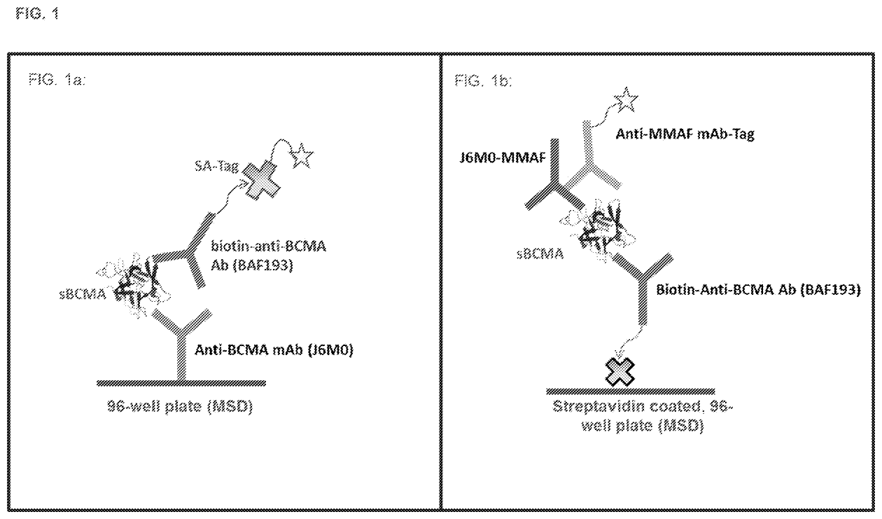

Exemplary assays for detecting sBCMA are demonstrated in FIG. 1. These assays are further described herein and in Example 1.

FIG. 1a demonstrates one exemplary method for the detection of free sBCMA (sBCMA not bound to a BCMA antigen binding protein). In one exemplary embodiment, free sBCMA is detected using the methods described in Example 1.

FIG. 1b demonstrates one exemplary method for the detection of sBCMA bound to a BCMA antigen binding protein. In one exemplary embodiment, sBCMA bound to a BCMA antigen binding protein is detected using the methods described in Example 1.

### Kits

In one embodiment, a kit for the treatment of cancer is provided, comprising a means (e.g. reagents) for determining the level of sBCMA in a sample from a patient, e.g. a human serum sample. In one embodiment, the means for determining the level of sBCMA in a sample comprises a capture antibody and/or a detection antibody that binds to sBCMA in the sample and which contains a detection moiety. Kits may include any means described herein for detection of sBCMA in a sample.

Sequence Listing

| | |
|---|---|
| SEQ ID NO: 1 | NYWMH |
| SEQ ID NO: 2 | ATYRGHSDTYYNQKFKG |
| SEQ ID NO: 3 | GAIYDGYDVLDN |
| SEQ ID NO: 4 | SASQDISNYLN |
| SEQ ID NO: 5 | YTSNLHS |
| SEQ ID NO: 6 | QQYRKLPWT |
| SEQ ID NO: 7 | QVQLVQSGAEVKKPGSSVKVSCKASGGTFSNYWMHWVRQAPGQ |
| | GLEWMGATYRGHSDTYYNQKFKGRVTITADKSTSTAYMELSSLR |
| | SEDTAVYYCARGAIYDGYDVLDNWGQGTLVTVSS |
| SEQ ID NO: 8 | DIQMTQSPSSLSASVGDRVTITCSASQDISNYLNWYQQKPGKAPK |
| | LLIYYTSNLHSGVPSRFSGSGSGTDFTLTISSLQPEDFATYYCQQYR |
| | KLPWTFGQGTKLEIKR |
| SEQ ID NO: 9 | QVQLVQSGAEVKKPGSSVKVSCKASGGTFSNYWMHWVRQAPGQ |
| | GLEWMGATYRGHSDTYYNQKFKGRVTITADKSTSTAYMELSSLR |
| | SEDTAVYYCARGAIYDGYDVLDNWGQGTLVTVSSASTKGPSVFP |
| | LAPSSKSTSGGTAALGCLVKDYFPEPVTVSWNSGALTSGVHTFPA |
| | VLQSSGLYSLSSVVTVPSSSLGTQTYICNVNHKPSNTKVDKKVEP |
| | KSCDKTHTCPPCPAPELLGGPSVFLFPPKPKDTLMISRTPEVTCVV |
| | VDVSHEDPEVKFNWYVDGVEVHNAKTKPREEQYNSTYRVVSVL |
| | TVLHQDWLNGKEYKCKVSNKALPAPIEKTISKAKGQPREPQVYTL |
| | PPSRDELTKNQVSLTCLVKGFYPSDIAVEWESNGQPENNYKTTPP |
| | VLDSDGSFFLYSKLTVDKSRWQQGNVFSCSVMHEALHNHYTQKS |
| | LSLSPGK |
| SEQ ID NO: 10 | DIQMTQSPSSLSASVGDRVTITCSASQDISNYLNWYQQKPGKAPK |
| | LLIYYTSNLHSGVPSRFSGSGSGTDFTLTISSLQPEDFATYYCQQYR |
| | KLPWTFGQGTKLEIKRTVAAPSVFIFPPSDEQLKSGTASVVCLLNN |
| | FYPREAKVQWKVDNALQSGNSQESVTEQDSKDSTYSLSSTLTLSK |
| | ADYEKHKVYACEVTHQGLSSPVTKSFNRGEC |
| SEQ ID NO: 11 | MLQMAGQCSQNEYFDSLLHACIPCQLRCSSNTPPLTCQRYCNASV |
| | TNSVKGTNAILWTCLGLSLIISLAVFVLMFLLRKINSEPLKDEFKNT |
| | GSGLLGMANIDLEKSRTGDEIILPRGLEYTVEECTCEDCIKSKPKV |
| | DSDHCFPLPAMEEGATILVTTKTNDYCKSLPAALSATEIEKSISAR |

### EXAMPLES

The following examples illustrate various non-limiting aspects of this invention.

### Methods:

In a dose escalation clinical study, 38 subjects were treated with J6M0-MMAF (Part1). The doses ranged from 0.03 mg/kg up to 4.60 mg/kg (0.03 mg/kg, 0.06 mg/kg, 0.12 mg/kg, 0.24 mg/kg, 0.48 mg/kg, 0.96 mg/kg, 1.92 mg/kg, 2.5 mg/kg, 3.4 mg/kg, and 4.6 mg/kg). An expansion cohort of 35 subjects followed at a dose of 3.40 mg/kg (Part2). An overall response rate (ORR) of 60% (21/35; 95% CI 42.1-76.1) by IMWG criteria was demonstrated. Levels of circulating soluble BCMA (sBCMA) were followed during these studies. Soluble BCMA was measured in serum samples collected at pre- and post-infusion of J6M0-MMAF. Immunoassays (FIG. 1) were used to determine the levels of free sBCMA (FIG. 1a) and J6M0-MMAF-bound sBCMA (FIG. 1b).

Free sBCMA Assay Protocol (FIG. 1a)
1.1. Preparation of Anti-BCMA Antibody Coated Plates (3 μg/ml)

1. An appropriate volume (˜3 mL per plate) of capture coating antibody was prepared—anti-BCMA antibody (J6M0) at 3 μg/mL in DPBS.

2. 25 μL of the coating solution was dispensed to each well of the 96-well high bind MSD plate (MSD Standard Streptavidin-Coated 96-well plate—Meso Scale Discovery Cat # L11SA).

3. The MSD plate was covered with a plate sealer and incubated in a 2-8° C. refrigerator on a flat surface overnight (18 hr±3 hr).

1.2. Preparation of huBCMA Standard Curve

1. On the day of the assay, a 12-point standard curve of known amounts of BCMA, ranging from 1000 ng/ml to 0 ng/ml, was prepared in BCMA-depleted serum.

1.3. Transfer of Samples onto Plate

1. The MSD plate was washed 3× with wash buffer (1×PBST).

2. 25 μL of diluted standard curve and test samples were transferred to the MSD assay plate. All samples were tested in duplicate.

3. The samples were incubated for 1 hour (±5 min) on a shaker at a speed of approximately 600 rpm.

### 1.4. Preparation of Detection Antibody and Reporter

1. Ten minutes prior to the end of sample incubation, detection antibody (Biotinylated Anti-BCMA pAB—R&D systems #BAF193) was prepare at 3 μg/mL in antibody diluent as follows:

180 μL of BAF193 (50 μg/mL) was added to 184, Streptavidin Sulfo TAG (0.5 mg/mL) (Meso Scale Discovery #R32AD-1)+2802 μL of antibody diluent (1% BSA/1×DPBS).

2. The MSD plate was washed 3× with wash buffer (1×PBST).

3. 25 μL of detection antibody solution was added into each well of the assay plate.

4. The plates were covered and and placed on a shaker set at approximately 600 rpm at room temperature for 1 hour (±5 min).

### 1.5. Read Buffer

1. 1×MSD read buffer was prepared as follows:

5 mL of 4×MSD read buffer T (Meso Scale Discovery #R92TC-1) plus 15 mL of Milli-Q water.

2. The MSD plate was washed 3× with wash buffer (1×PBST).

3. 1504, of 1× read buffer was transferred into to each well of the assay plate.

4. The MSD assay plate was read immediately by using the MSD Sector Imager 6000 (Meso Scale Discovery).

Bound sBCMA Assay Protocol (sBCMA Bound to J6M0-MMAF) (FIG. 1b)

### 2.1 Plate Coating

1. Biotinylated Anti-BCMA pAb (R&D Systems #BAF193) was diluted to a concentration of 50 μg/mL in 1×PBS.

2. 180 μL of Biotin-BAF193 (50 μg/mL) was added to 5820 μL of coating buffer (25 mM Hepes/0.015% Triton X-100).

3. 50 μL of the coating solution was dispensed into each well of an MSD Standard Streptavidin-Coated 96-well plate (Meso Scale Discovery Cat # L11SA).

4. The plate was covered and incubated in a 2-8° C. refrigerator overnight.

### 2.2. Blocking the Plate

1. The MSD plate was washed with wash buffer (0.1% DPBST).

2. 150 μl of 3% Blocking buffer (MSD Blocker A—Meso Scale Discovery #R93BA-4) was transferred into each well on the plate.

3. The plate was covered and placed on a plate shaker (approximately 600 rpm) at room temperature for 1-2 hour.

### 2.3. Preparation of Standard Curve, Samples and QCs for Analysis

1. A 9-point standard curve of known amounts of BCMA/J6M0-MMAF, ranging from 200 ng/ml BCMA/20 μg/mL J6M0-MMAF to 0 ng/ml BCMA/0 μg/mL J6M0-MMAF, was prepared in BCMA-depleted serum.

2.4. Transfer of Samples onto the Assay Plate

1. The MSD plate was washed with wash buffer (0.1% DPBST).

2. 25 μL of diluted standard curve and test samples were transferred to the MSD assay plate. All samples were tested in duplicate.

3. The assay plate was covered and place on plate shaker (approximately 600 rpm) at room temperature and incubated for 2 hours±5 minutes.

2.5. Detecting Antibody (sTag Anti-Auristatin MMAF Antibody)

1. An sTag-ruthenium anti-auristatin MMAF antibody was dilute to 1 μg/mL in antibody diluent (1% BSA/1×DPBS). (12 μL of sTag anti-auristatin MMAF (0.5 mg/mL) was added to 5988 mL of antibody diluent).

2. The MSD plate was washed with wash buffer (0.1% DPBST).

3. 25 μL of the sTag anti-auristatin solution was added to each well on the plate.

4. The plate was covered and incubated at room temperature with shaking (600 rpm) for 1 hour±5 minutes.

### 2.6. Plate Reading

1. After ˜1 hour, the plate was wash with wash buffer (0.1% DPBST).

2. 150 μL of 2×MSD Read Buffer T (Meso Scale Discovery #R92TC-1) was added to each well of the plate.

3. The plate was read within 10 minutes on the MSD SECTOR Imager 6000 (Meso Scale Discovery).

### Example 1

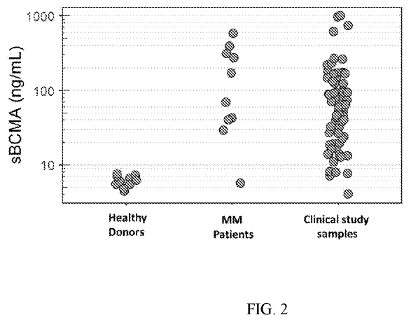

Soluble BCMA was measured in serum from healthy donors (n=10), from multiple myeloma (MM) patients (n=10), and from samples from patients enrolled in the clinical study described herein. The levels of baseline sBCMA observed in in the samples from the clinical study were comparable to those observed in multiple myeloma patients and showed higher levels than those observed in healthy donor serum (FIG. 2). Examination of circulating sBCMA revealed high sBCMA levels, with a baseline median concentration of free sBCMA of 58 ng/mL across all doses (n=68; range 4 ng/mL to >1000 ng/mL).

### Example 2

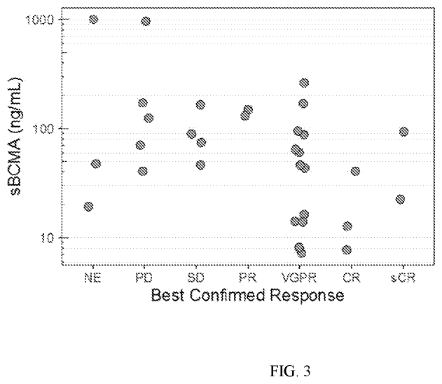

Soluble BCMA was measured at baseline (prior to infusion) in subjects from the dose expansion cohort (Part 2). FIG. 3 demonstrates the best confirmed response obtained for each patient relative to the baseline measures of sBCMA. The levels of baseline sBCMA were generally lower in the non-responding patients (81 ng/mL, n=12; compared to 43 ng/mL, n=19). High baseline sBCMA levels were also found in responders with levels up to 262 ng/mL.

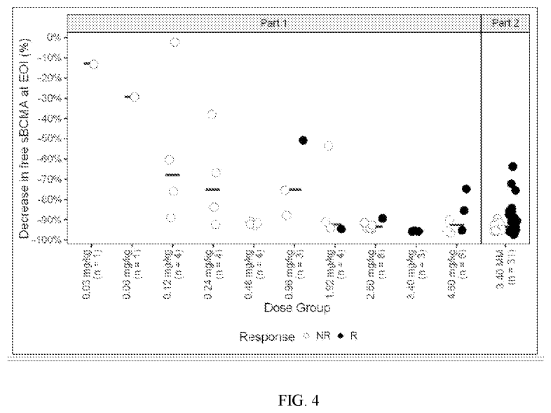

The binding of J6M0-MMAF to sBCMA was measured by comparing the post-infusion levels of free sBCMA measured 60 minutes after the start of infusion to those found at pre-infusion. FIG. 4 demonstrates that the reduction in free sBCMA appeared to be related to the dose level administered, and doses above 1.92 mg/kg consistently achieved a greater than 90% reduction of free sBMCA (percentage change from baseline). Points are colored by whether the patient had a best clinical response of PR or better (R) or was a non-responder (NR). The average percentage decrease for each dose group is shown as a horizontal black line. Part 1 is the dose escalation cohort and Part 2 is the dose expansion cohort.

At higher dose levels, it was found that J6M0-MMAF binds a large fraction of sBCMA, and responses to J6M0-MMAF were observed in 60% of dose expansion subjects with either low or high baseline sBCMA. Higher baseline sBCMA in non-responders compared to responders because J6M0-MMAF being bound by soluble BCMA as evidenced by doses above 1.92 mg/kg achieving >90% reduction of free sBCMA.

1. A method for determining prognosis of cancer in a patient, comprising:
(a) obtaining a sample from the patient; and
(b) testing the sample for the presence of soluble BCMA expression;
wherein, if the patient has expression of soluble BCMA, then the patient's prognosis is poor.

2. The method of claim 1, wherein the expression of soluble BCMA is a high level of soluble BCMA expression.

3. A method of predicting a patient's response to treatment with a BCMA antigen binding protein comprising: (a) obtaining a sample from a patient; and (b) testing the sample for the level of soluble BCMA expression, wherein if the patient expresses a high level of soluble BCMA, the patient is predicted to not respond to treatment with a BCMA antigen binding protein.

4. The method of claim 3, wherein the patient is a multiple myeloma patient, and wherein the soluble BCMA expression is high when the level of soluble BCMA expression is greater than about 40 ng/ml or greater than 50 ng/ml.

5. The method of claim 4, wherein the multiple myeloma patient is predicted to not respond to treatment with a BCMA antigen binding protein comprising a VH comprising an amino acid sequence set forth in SEQ ID NO:7; a VL comprising an amino acid sequence set forth in SEQ ID NO:8, and wherein the antibody is conjugated to MMAF.

6. A method for treating cancer in a patient in need thereof, comprising:
(a) obtaining a sample from the patient; and
(b) testing the sample for expression of soluble BCMA; and
(c) if the subject expresses soluble BCMA, administering to the patient an effective amount of a BCMA antigen binding protein.

7. A method for treating cancer in a patient in need thereof, comprising:
(a) obtaining a sample from the patient; and
(b) testing the sample for the level of soluble BCMA expression; and
(c) if the patient has a high level of soluble BCMA expression, administering to the patient an effective amount of a BCMA antigen binding protein.

8. The method of claim 7, wherein the level of soluble BCMA expression in the sample is high when the level of soluble BCMA is above about 10 ng/ml.

9. A method for selecting the dose of a BCMA antigen binding protein for treating cancer in a patient in need thereof, comprising:
(a) obtaining a sample from the patient;
(b) testing the sample for the level of soluble BCMA expression; and
(c) if the patient has a low level of soluble BCMA expression, treating the patient with a low dose of a BCMA antigen binding protein; or if the patient has a high level soluble BCMA expression, treating the patient with a high dose of a BCMA antigen binding protein.

10. (canceled)

11. The method of claim 1, wherein the patient is human patient.

12. The method of claim 1, wherein the cancer is selected from the group consisting of multiple myeloma, lymphoma, chronic lymphocytic leukemia, non-Hodgkin's lymphoma, follicular lymphoma, and diffuse large B-cell lymphoma.

13. The method of claim 1, wherein the cancer is multiple myeloma, lymphoma.

14. The method of claim 1, wherein the sample is a serum or blood sample.

15. The method of claim 3, wherein the BCMA antigen binding protein is an antibody, an antibody fragment, a bispecific antibody, an antibody-drug-conjugate, a bispecific T-cell engager (BITE), or a chimeric antigen receptor T-cell (CAR-T).

16. The method of claim 15, wherein the BCMA antigen binding protein is a monoclonal antibody comprising a VH comprising an amino acid sequence set forth in SEQ ID NO:7; and a VL comprising an amino acid sequence set forth in SEQ ID NO:8, wherein the antibody is conjugated to MMAF.

17. The method of claim 6, wherein the patient is further treated with at least one additional anti-neoplastic agents.

18. The method of claim 17, wherein the at least one additional neoplastic agent is selected from the group consisting of an anti-PD1 antibody, an anti-ICOS antibody, and anti-OX40 antibody, an anti-CD38 antibody, a proteasome inhibitor, a thalidomide analog, and dexamethasone.

19.-26. (canceled)

27. A kit for the treatment of cancer with a BCMA antigen binding protein in a patient, comprising a means for determining the level of soluble BCMA in a sample from the patient.

28.-31. (canceled)

32. A method for treating cancer in a patient in need thereof, comprising determining if a patient expresses soluble BCMA, wherein if the patient is determined to express soluble BCMA, administering to the patient an effective amount of a BCMA antigen binding protein.

33. The method of claim 1, wherein the expression of soluble BCMA is a high level of soluble BCMA.
